(19) United States
(12) Patent Application Publication
(10) Pub. No.: US 2008/0206441 A1
(43) Pub. Date: Aug. 28, 2008

(54) Ion Beam Etching a Surface of an Implantable Medical Device

(75) Inventor: Mike Krivoruchko, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/679,276

(22) Filed: Feb. 27, 2007

(51) Int. Cl. A61L 33/00

(52) U.S. Cl. 427/2.25

(57) ABSTRACT

An implantable medical device capable of delivering therapeutic substances includes a roughened surface formed by ion beam etching. Ionized gas particles are shot at the medical device at high velocity to ablate portions of the surface of the medical device. The medical device, or a portion thereof, can be coated with a coating containing a therapeutic substance or substances, a polymer, or a combination of therapeutic substances and polymer. The coating can be made of one or more layers and the various layers can include different therapeutic substances, polymers, or combinations of therapeutic substances and polymers. The roughened surface has a greater surface area than a smooth surface, providing a better mechanical hold for the coating, thereby improving coating retention or therapeutic substance elution.

### FIELD OF THE INVENTION

This invention relates generally to methods for forming surface features of implantable medical devices. More particularly, the present invention is directed to methods of using ion beam etching to roughen the surface of implantable medical devices, such as stents and grafts, with numerous surface features such as pits, protrustion, depressions to create controllable topology to improve stent clinical performance and efficacy.

### BACKGROUND OF THE INVENTION

Endovascular stents are coated frequently with a polymer that contains one or more therapeutic substances within a polymeric matrix to improve the efficacy of the stents. These substances are eluted from the stent coating to the tissue bed surrounding the implanted stent. The effectiveness of these therapeutic substances is generally improved because localized levels of medication may be higher and potentially more successful than orally or intravenously delivered drugs, which are distributed throughout the body rather than concentrated at the location of most need. Drugs released from tailored stent coatings may have controlled, timed-release qualities, eluting their bioactive agents over hours, weeks or even months. A common solvent or a pair of solvents may be used to dissolve drugs and polymers, including copolymers, terpolymers or polymer blends. Then the drug-polymer solution is sprayed or dipped on the stent. Upon drying, the drug-polymer coating is formed on the stent surface.

Polymer matrices containing the compounds or the therapeutic compounds themselves, must be reliably attached to the stent to control delivery/elution of the pharmaceutical compounds, to maintain high quality during manufacturing of such a stent, and to prevent cracking or flaking of the drug-polymer coating when the stent is deployed. Problems may arise in getting coatings to adhere to stents, particularly stents made of stainless steel. Most coronary stents are made of stainless steel or tantalum and are finished by electrochemical polishing for surface smoothness. A smooth surface is desirable because early research has shown that a stent with a rough surface results in more platelet cell adhesion, thrombus, inflammation, and restenosis than a smoothly polished stent. The smooth surface may pose a challenge to the coating, however. Due to the very different nature of the polymer/therapeutic agent and the metallic substrate, organics/polymers do not easily adhere to the metallic substrate. If the coating does not adhere well to the metal surface, it may cause problems such as coating delamination, irregular drug release profiles, or embolism caused by broken and detached debris from the coating.

The coating may crack or fall off during assembly, packaging, storage, shipping, preparation and sterilization prior to deployment unless effectively adhered to the stent framework. Degradation of the polymer coating may occur with prolonged exposure to light and air, as the constituents of the drug polymer may oxidize or the molecular chains may scission. Although degradation of the polymer coating is of major concern, it is imperative that the adhesion strength of the coating be greater than the cohesive strength of the polymeric matrix to avoid any loss of the coating.

Organic compounds and polymeric coatings have a tendency to peel or separate from an underlying metallic stent because of low adhesion strength typically found between organics and metals. Many organics are non-polar or have limited polarization, reducing their ability to stick to the metal stent framework. Temperature excursions of the coated stent and the difference in thermal expansion coefficients between the metal and the coating may contribute to the fatigue and failure of the bond. Materials that are optimal for drug compatibility and elution may not, in and of themselves, provide sufficient adhesion to a metal substrate. A method to improve the adhesion between a drug-polymer coating and a metallic stent, while retaining the therapeutic characteristics of the drug-polymer stent, would be beneficial.

### BRIEF SUMMARY OF THE INVENTION

An implantable medical device capable of delivering therapeutic substances from a surface or a coating is provided, along with a method of preparing the device. In comparison to a conventional implantable medical device, the implantable medical device of the present invention can better retain a coating and allow a greater total amount of coating to be carried by the device, thereby allowing for greater amounts of therapeutic substances to be delivered from the device.

In an embodiment of a method of manufacture within the present invention, ion beam etching is used on a designated region of a surface or the entire surface of the implantable medical device to selectively roughen or modify the entire surface of the implantable medical device. In ion beam etching, ionized gas particles, such as argon, helium, oxygen are shot at the medical device at high velocity to ablate portions of the surface of the medical device. Ion beam etching can achieve surface features with very high aspect ratios, depth/width, to create very deep topological features.

In various embodiments, the medical device, or a portion thereof, can be coated with a coating containing a therapeutic substance or substances, a polymer, or a combination of therapeutic substances and polymer. The coating can be made of one or more layers and the various layers can include different therapeutic substances, polymers, or combinations of therapeutic substances and polymers. The roughened surface has a greater surface area and more bonding sites than a smooth surface, providing a better mechanical hold for the coating, thereby improving coating retention.

The type of implantable medical device treated in accordance with the various embodiments of the invention may vary. For example, the implantable device may be a stent or a graft.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective view of an example of an exemplary stent of an embodiment of the present invention.

FIG. 2 is a sectional view of a stent strut of the stent of FIG. 1 showing a roughened outer surface of the strut.

FIG. 3 is a sectional view of a stent strut of the stent of FIG. 1 showing roughened outer and inner surfaces of the strut.

FIG. 4 is a sectional view of a stent strut of the stent of FIG. 1 showing roughened outer, inner and side surfaces of the strut.

FIG. 5 is a sectional view the strut of FIG. 2 with a coating or therapeutic agent covering the roughened outer surface of the strut.

FIG. 6 is a sectional view the strut of FIG. 3 with a coating or therapeutic agent covering the roughened outer and inner surfaces of the strut.

FIG. 7 is a sectional view the strut of FIG. 4 with a coating or therapeutic agent covering the roughened outer, inner, and side surfaces of the strut.

FIG. 8 is a sectional view the strut of FIG. 2 with a two layers of coating covering the roughened outer surface of the strut.

FIG. 9 is a sectional view the strut of FIG. 3 with a two layers of coating covering the roughened outer and inner surfaces of the strut.

FIG. 10 is a sectional view the strut of FIG. 4 with a two layers of coating covering the roughened outer, inner, and side surfaces of the strut.

FIG. 11 is a schematic representation of an ion beam etching apparatus that can be used to roughen the surface of an implantable medical device.

### DETAILED DESCRIPTION OF THE INVENTION

Specific embodiments of the present invention are now described with reference to the figures, where like reference numbers indicate identical or functionally similar elements.

The present invention provides a method for treating the surface of an implantable medical device, such as a stent or graft, which are often referred to as endoprostheses. Beyond stents and grafts, however, other implantable medical devices, such as artificial joints, bones, pacemakers, and the like, may be made in accordance with the methods of the present invention and used for drug delivery. In the discussion below, the example of a stent is provided. Practitioners will appreciate, however, that the methods and structures of the present invention are not limited to a stent, but rather extend to all implantable devices having a metallic surface upon which a coating can be deposited.

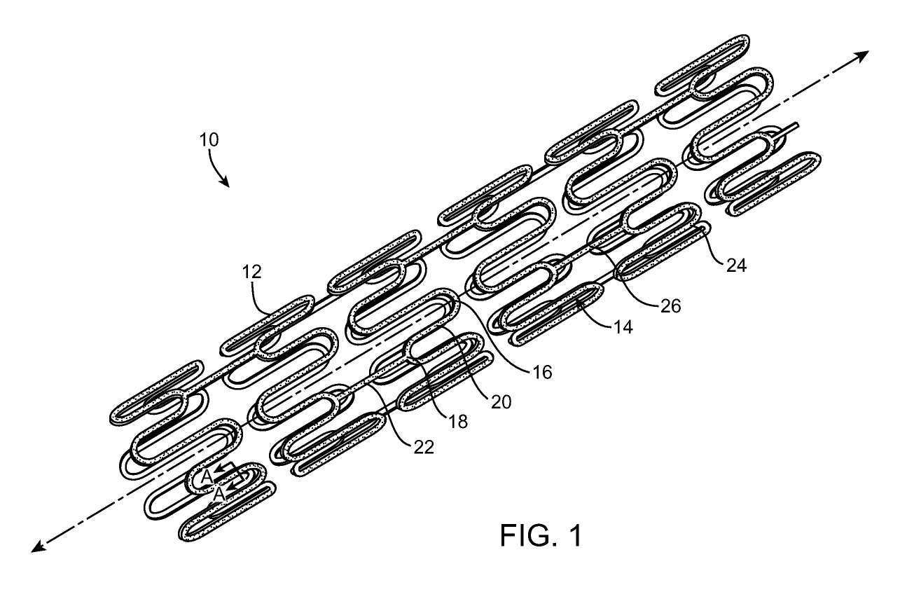

FIG. 1 illustrates an exemplary stent 10 in accordance with an embodiment of the present invention. Stent 10 is a patterned tubular device that includes a plurality of radially expandable cylindrical rings 12. Cylindrical rings 12 are formed from struts 14 formed in a generally sinusoidal pattern including peaks 16, valleys 18, and generally straight segments 20 connecting peaks 16 and valleys 18. Connecting links 22 connect adjacent cylindrical rings 12 together. In FIG. 1, connecting links 22 are shown as generally straight links connecting a peak 16 of one ring 12 to a valley 18 of an adjacent ring 12. However, connecting links 22 may connect a peak 16 of one ring 12 to a peak 16 of an adjacent ring, or a valley to a valley, or a straight segment to a straight segment. Further, connecting links 22 may be curved. Connecting links 22 may also be excluded, with a peak 16 of one ring 12 being directly attached to a valley 18 of an adjacent ring 12, such as by welding, soldering, or the manner in which stent 10 is formed, such as by etching the pattern from a flat sheet or a tube. It will be appreciated by those skilled in the art that stent 10 of FIG. 1 is merely an exemplary stent and that stents of various forms and methods of fabrication can be used. For example, in a typical method of making a stent, a thin-walled, small diameter metallic tube is cut to produce the desired stent pattern, using methods such as laser cutting or chemical etching. The cut stent may then be descaled, polished, cleaned and rinsed. Some examples of methods of forming stents and structures for stents are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated in its entirety by reference herein.

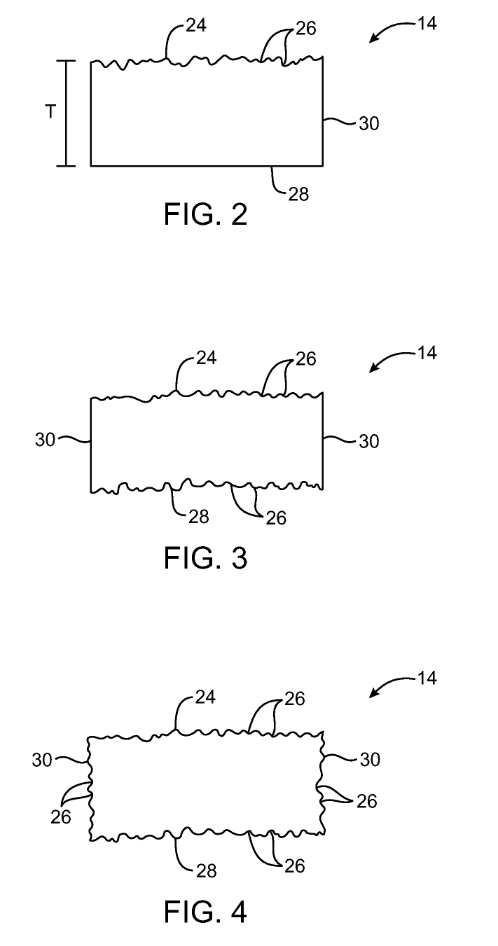

Stent 10 shown in FIG. 1 includes an outer surface 24 showing pits or surface features 26. Nanometer or micrometer scale pits/features 26 are formed on surface 24 using ion beam etching, as will be explained in more detail below. FIG. 2 is a cross-sectional view taken at A-A through a portion of strut 14 of stent 10. Strut 14 has a suitable thickness T between the stent outer surface 24 and an inner surface 28. Typically, thickness T may be in the range of approximately 50 μm (0.002 inches) to 200 μm (0.008 inches). A cross-sectional view of connecting links 22 may be similar to strut 14, or may be different. For example, a thickness of connecting links 22 may be different than strut 14 of cylindrical rings 12 for variable flexibility between the rings 12 and connecting links 22. A specific choice of thickness for struts 14 and links 22 depends on several factors, including, but not limited to, the anatomy and size of the target lumen. Further, connecting links 22 may or may not include pits/features 26 on a surface thereof.

Typical materials used for stent 10 are metals or alloys, examples of which include, but are not limited to, stainless steel, “MP35N,” “L605” nickel titanium alloys such as Nitinol (e.g., ELASTINITE® by Advanced Cardiovascular Systems, Inc., Santa Clara, Calif.), tantalum, platinum-iridium alloy, gold, magnesium, or combinations thereof. “MP35N” and “L605” are trade names for alloys of cobalt chromium and nickel. MP35N is available from standard Press Steel Co., Jenkintown, Pa. “MP35N” consists of 35% cobalt, 35% nickel, 20% chromium, and 10% molybdenum.

In accordance with the present invention, ionized gas particles, such as argon, helium, and oxygen are shot at a designated region of the surface of stent 10 or other implantable medical device. For example, the ionized gas particles may be directed to the entire outer surface 24, the entire inner surface 28, side surfaces 30, all surfaces, or just a portion of the inner surface 28, outer surface 24, and/or side surfaces 30. The ionized gas particles contact the surface with sufficient energy to vaporize the material on the surface of the implantable medical device, creating numerous pits or features 26, the combined effect of which is a rough surface having increased surface area.

Referring back to FIG. 2, numerous pits/features 26 have been formed on outer surface 24 of stent 10 using ion beam etching. However, inner surface 28 has not been roughened. FIG. 3 is a sectional view also taken at A-A of FIG. 1 of strut 14 illustrating an alternative embodiment with pits/features 26 formed on both outer surface 24 and inner surface 28 of stent 10. Similarly, FIG. 4 is an alternative embodiment showing a sectional view taken at A-A of FIG. 1 illustrating pits/features 26 formed on outer surface 24, inner surface 28, and side surfaces 30 of stent 10. The size and distribution of pits or surface features 26 of the roughened surface(s) may be controlled by variables affecting the ionized gas or by selection of the ion beam etching apparatus, as further discussed below. The desired feature size would be on the range of tens of nanometer to several microns.

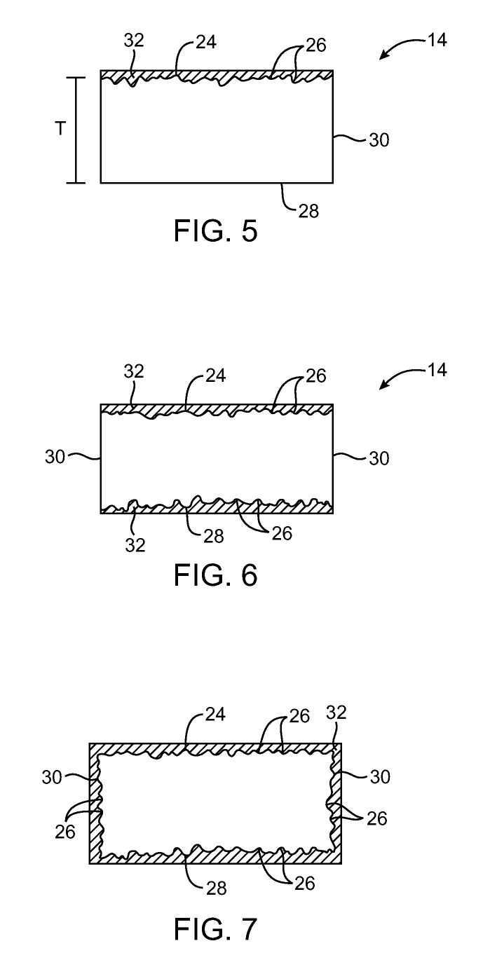

FIG. 5 illustrates a coating 32 covering outer surface 24 of stent 10 shown in FIG. 2. Coating 32 may be, for example, a therapeutic compound or a polymeric coating that contains a therapeutic substance. Coating 32 fills and covers pits/features 26. Pits/features 26 provide a mechanical hold for coating 32 and help to prevent coating 32 from slipping or peeling off of the implantable device. In addition, because pits/features 26 increase the surface area of surface 24, the amount of coating that can be put onto surface 24 of stent 10 is increased. The greater amount of coating allows the implantable device to carry more therapeutic substance, so more medicine can be delivered from the implantable device in situ. Similarly, FIG. 6 shows coating 32 covering inner surface 28 and outer surface 24 of strut 14 shown in FIG. 3. FIG. 7 shows coating 32 covering inner surface 28, outer surface 24, and side surfaces 30 of strut 14 shown in FIG. 4. It will be appreciated by one skilled in the art that coating 32 need not only cover the pitted surfaces of strut 14. For example, coating 32 in FIG. 5 may cover inner surface 28, side surfaces 30, and outer surface 24 even though outer surface 24 is the only surface with pits/features 26. It will be appreciated that in such an embodiment, pits/features 26 of outer surface 24 would improve retention of coating 32 even though pits/features 26 are only on outer surface 24. It will further be appreciated that connecting links 22 may or may not be coated similar to struts 14.

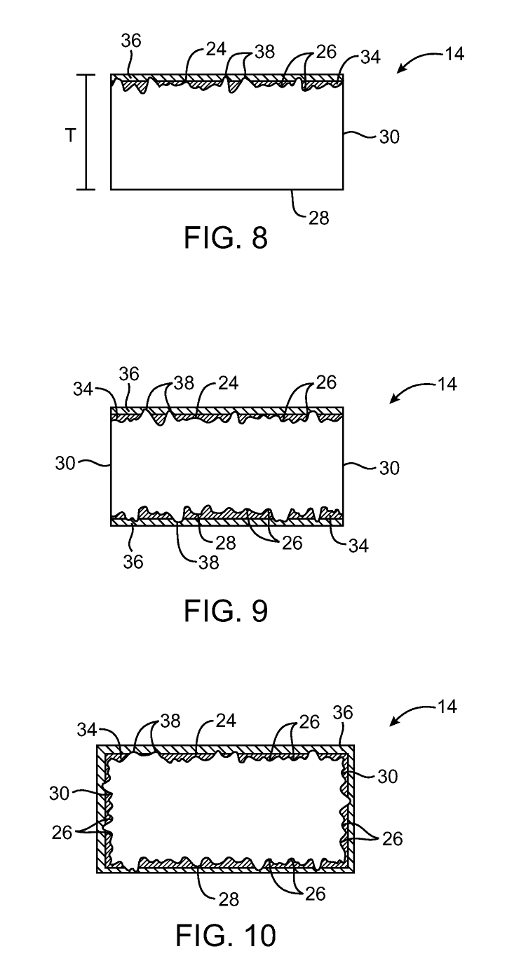

In alternative embodiments illustrated in FIGS. 8-10, the pitted surface(s) can allow two or more layers of a coating to adhere to one or all of the surfaces. In FIG. 8, a first coating layer 34 is coated onto outer surface 24 and then covered with a second coating layer 36. First coating layer 34 partially fills the deeper area of pits/features 26, but portions 38 of surface 24 extend above first coating layer 34. Second coating layer 36 can adhere to both first coating layer 34 and portions 38 of outer surface 24. FIGS. 9 and 10 show the same multiple coating layers coating multiple surfaces of strut 14. Further, one skilled in the art would appreciate that more than two coating layers could be utilized, if appropriate.

The multiple coating layers of FIGS. 8-10 can be used to achieve different therapeutic substance release profiles. For example, if it is desired to release two therapeutic substances sequentially, two layers as illustrated in FIGS. 8-10 can be used. For example, a therapeutic substance in the second coating layer 36 will be released first, as second coating layer 36 dissolves, and a therapeutic substance in first coating layer 34 will be released after second coating layer 36 has wholly or partially dissolved. This release profile is sometimes referred to as a “late burst”, especially if the second therapeutic substance to be released (that in first coating layer 34) is in a highly soluble form, for instance, pure crystalline form.

Methods of coating a stent or other implantable medical device with one or more therapeutic substances, or with a polymer containing one or more therapeutic substances are well-known. For example, one or more therapeutic substances can be added to stent 10 by dissolving or mixing the therapeutic substances in a solvent and applying the therapeutic substance and solvent mixture to stent 10. To cover stent 10 with a polymer containing the therapeutic substance or substance combination, a solution of the polymeric material and one or more therapeutic substances are mixed, often with a solvent, and the polymer mixture is applied to the implantable device. Stent 10 can also be coated with a polymer that does not contain a therapeutic substance, for example, to form a sealant layer over an underlying layer, which does contain a therapeutic substance.

Methods of applying the therapeutic substance, polymer, or therapeutic substance and polymer mixture to stent 10 include, but are not limited to, immersion, spray-coating, sputtering, and gas-phase polymerization. Immersion, or dip-coating, entails submerging the entire stent 10, or an entire section of stent 10, in the mixture. Stent 10 is then dried, for instance in a vacuum or oven, to evaporate the solvent, leaving the therapeutic substance or therapeutic substance and polymer coating on the stent. Similarly, spray-coating requires enveloping the entire stent, or an entire section of the stent, in a large cloud of the mixture, and then allowing the solvent to evaporate, to leave the coating. Sputtering typically involves placing a polymeric coating material target in an environment, and applying energy to the target such that polymeric material is emitted from the target. The polymer emitted deposits onto the device, forming a coating. Similarly, gas phase polymerization typically entails applying energy to a monomer in the gas phase within a system set up such that the polymer formed is attracted to a stent, thereby creating a coating around the stent.

The polymer used for coating stent 10 is typically either bioabsorbable or biostable. A bioabsorbable polymer bio-degrades or breaks down in the body and is not present sufficiently long after implantation to cause an adverse local response. Bioabsorbable polymers are gradually absorbed or eliminated by the body by hydrolysis, metabolic process, bulk, or surface erosion. Examples of bioabsorbable, biodegradable materials include but are not limited to polycaprolactone (PCL), poly-D, L-lactic acid (DL-PLA), poly-L-lactic acid (L-PLA), poly(lactide-co-glycolide), poly(hydroxybutyrate), poly(hydroxybutyrate-co-valerate), polydioxanone, polyorthoester, polyanhydride, poly(glycolic acid), poly(glycolic acid-cotrimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, polyiminocarbonates, and aliphatic polycarbonates. Biomolecules such as heparin, fibrin, fibrinogen, cellulose, starch, and collagen are typically also suitable. Examples of biostable polymers include Parylene®, Parylast®, polyurethane (for example, segmented polyurethanes such as Biospan®), polyethylene, polyethlyene terephthalate, ethylene vinyl acetate, silicone and polyethylene oxide.

Therapeutic substances can include, but are not limited to, antineoplastic, antimitotic, antiinflammatory, antiplatelet, anticoagulant, anti fibrin, antithrombin, antiproliferative, antibiotic, antioxidant, and antiallergic substances as well as combinations thereof. Examples of such antineoplastics and/or antimitotics include paclitaxel (e.g., TAXOL® by Bristol-Myers Squibb Co., Stamford, Conn.), docetaxel (e.g., Taxotere® from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack N.J.), and mitomycin (e.g., Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.). Examples of such cytostatic or antiproliferative agents include angiopeptin, angiotensin converting enzyme inhibitors such as captopril (e.g., Capoten® and Capozide® from Bristol-Myers Squibb Co., Stamford, Conn.), cilazapril or lisinopril (e.g., Prinivil® and Prinzide® from Merck & Co., Inc., Whitehouse Station, N.J.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include alpha-interferon, genetically engineered epithelial cells, and dexamethasone. In other examples, the therapeutic substance is a radioactive isotope for implantable device usage in radiotherapeutic procedures. Examples of radioactive isotopes include, but are not limited to, phosphoric acid (H₃P³²O₄), palladium (Pd¹⁰³), cesium (Cs¹³¹), and iodine (I¹²⁵). While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions.

The ion beam etching process for forming pits/features 26 on the surface(s) of stent 10 will now be described. In ion beam etching, an ion beam is irradiated onto a specimen (target) in a high vacuum and physically sputters away the irradiated surface atoms. In case the radius of the ion beam is reduced by electronic lens system, a maskless etching can be performed by sweeping the ion beam on the specimen. In case positive ions are used as the etching agent, surface charge-up of the specimen can be prevented by neutralizing the accelerated or bombarded positive ions with electrons. Ion beam etching allows accurate and directional etching to be achieved, since the etching agent collides with the target in one direction.

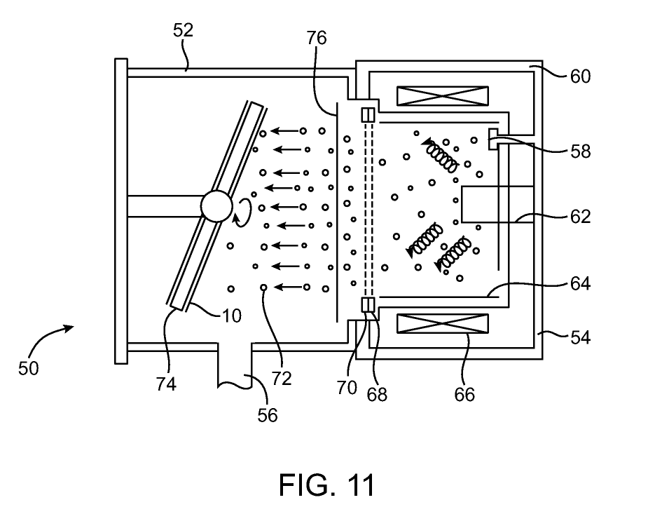

FIG. 11 shows a schematic representation of an example of an ion beam etching apparatus 50 for roughening the surface of a stent according to the present invention. Ion beam etching apparatus 50 includes a work chamber 52 and a source chamber 54. Source chamber 54 includes and ion gun chamber 60 for generating a plasma to produce, for example, argon ions. Work chamber 52 is evacuated by a vacuum pump through an exhaust pipe 56 so that the pressure inside work chamber 52 may be in the range of 10⁻³ to 10⁻⁷ Torr. Argon or another inert gas, such as helium, is introduced into ion gun chamber 60 through a port 58 at a predetermined flow rate. An electrical field created a cathode 62 and anodes 64 in ion gun chamber 60 ionizes the argon atoms. A coil 66 disposed around ion gun chamber 60 generates a magnetic field crossing the electric field so that the emitted electron makes a long stay in ion gun chamber 60, to enhance the ionization probability of the argon atom, thereby generating a plasma. Argon ions 72 produced in the plasma are led into work chamber 52 through grids 68 and 70 on the basis of a potential difference between the ion gun chamber 60 and work chamber 52, and impinge on stent 10 which is placed on a mandrel 74. Mandrel 74 is rotatable and an angle of incidence between mandrel 74 and the direction of the argon ions is variable. In order to prevent the surface of stent 10 from being charged with the argon ions, a neutralization filament 76 emits thermal electrons to neutralize electric charges on the surface of the stent 10. In an alternative embodiment, stent 10 may be placed into work chamber 52 as a flat sheet, in which case mandrel 74 would be replaced by a flat substrate holder.

Although a maskless ion beam etching process is possible, as described above, ion beam etching may require a mask to be applied to portions of the surface(s) to be roughened to create the pattern for etching. The portions of the surface(s) covered with a mask are not etched because the mask is etched instead. However, the exposed surfaces of the stent are etched. Possible masking methodologies include, but are not limited to, natural seeding, random seeding, and interval seeding. Natural seeding takes advantage of the natural variation in the strut material. Because of these variations, sputter yields vary and random spacing of the features occurs. Random seeding creates a random pattern of features when natural seeding fails. Feature density and distribution can be more closely controlled. Interval seeding can be used to create a uniformly spaced pattern on a material.

In general, the ion beam etching process to roughen the surface(s) of stent 10 may take place at any point in the stent manufacturing process, provided that subsequent processing does not remove the pitted surface desired in the completed stent. For example, the outer surface 24 may be roughed after stent 10 has been polished. In this case the ionized gas particles reach only outer surface 24 of stent 10, so inner surface 28 is protected and remains smooth. Alternatively, the ionized gas particles are shot at the stent material before the stent pattern is cut. In these cases, the roughened surface(s) may need to be preserved when the stent pattern is cut, descaled, and polished, if these processing operations will smooth the pitted surface more than is desired. To protect the roughened surface(s), a temporary protective coating, for example, a poly(vinyl alcohol) coating, can be applied to the outer surface of the stent before subsequent processing.

Although ion beam etching has been described herein, it is possible to roughen the surface(s) of a stent with reactive ion beam etching or chemically assisted ion beam etching. In ion beam etching (IBE), an inert gas, such as argon is used. Reactive ion beam etching (RIBE) is identical to ion beam etching except that reactive ions are incorporated in whole or in part in the etching ion beam. Thus, a reactive gas, such as oxygen, nitrogen or hydrogen, is provided to an ion source resulting in a reactive ion flux directed at a substrate. In chemically assisted ion beam etching (CAIBE), an inert gas is provided to the ion source, similar to ion beam etching. An ion flux is created that reacts with a reactive gas, such as chlorine, hydrogen, fluorine, prior to striking substrate.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of treating an implantable medical device comprising the steps of:
providing an implantable medical device having a first surface; and
applying ionized gas particles to a designated region of the first surface to remove material from said first surface, thereby forming numerous features or pits in the first surface.

2. The method of claim 1, wherein the implantable medical device is a stent.

3. The method of claim 2, wherein the first surface is an outer surface of the stent, the stent includes an inner surface opposite the outer surface, and further comprising polishing the inner surface of the stent.

4. The method of claim 2, wherein the first surface is an outer surface of the stent, the stent includes an inner surface opposite the outer surface, and further comprising the step of applying ionized gas particles to a designated region of the inner surface to remove material from the inner surface, thereby forming numerous features or pits in the inner surface.

5. The method of claim 1, further comprising the step of applying a first layer of a coating material over a portion of the designated region of the implantable medical device after forming said pits.

6. The method of claim 1, wherein said coating comprises a therapeutic substance.

7. The method of claim 5, further comprising applying a second layer of a coating material to the implantable medical device or portion thereof so as to cover at least a portion of the first layer.

8. The method of claim 7, wherein at least one of the first and second layers comprises a therapeutic substance.

9. The method of claim 1, further comprising the steps of descaling, polishing, cleaning and rinsing the medical device.

10. The method of claim 19, wherein the steps of descaling, polishing, cleaning an rinsing the medical device are accomplished prior to the step of applying ionized gas particles to the first surface.

11. A method of treating an implantable medical device comprising the steps of:
providing an implantable medical device having a first surface;
inserting the implantable medical device into an ion beam etching apparatus;
introducing a gas into an ion gun chamber of the ion beam etching apparatus;
ionizing the gas; and
impinging the ionized gas onto designated region of the first surface to remove material from the first surface, thereby forming numerous features or pits in the first surface.

12. The method of claim 11, wherein the gas is an inert gas.

13. The method of claim 12, wherein the gas is selected from the group consisting of argon, helium, neon, and xenon.

14. The method of claim 11, wherein the gas is a reactive gas.

15. The method of claim 14, wherein the gas is selected from the group consisting of oxygen, hydrogen, and nitrogen.

16. The method of claim 12, wherein prior to the step of impinging the designated region of the first surface, the ionized gas particles react with a reactive gas.

17. The method of claim 16, wherein the reactive gas is selected from the group consisting of chlorine and fluorine.
